(19) United States
(12) Patent Application Publication
(10) Pub. No.: US 2011/0009345 A1
(43) Pub. Date: Jan. 13, 2011

(54) Food ingredient comprising functional peptide

(76) Inventor: Henry Aoki, Acton, MA (US)

Correspondence Address: Lipsitz & McAllister, LLC, 755 MAIN STREET, MONROE, CT, 06468, US

(21) Appl. No.: 12/459,926

(22) Filed: Jul. 8, 2009

(51) Int. Cl. A61K 38/00; A23C 9/154

(52) U.S. Cl. 514/21.8; 426/580

(57) ABSTRACT

With efficient extracting of peptide from coffee bean, and by providing food ingredient in which small quantity of peptide is uniformly dispersed by harmless means or dispersing means, the peptide is easily orally ingested. That is, so much material and long time is required for extracting. And, quantity of novel ingredient of peptide obtainable by the extracting is very small, so it is difficult to use the ingredient as it is for material of medicine or food. So, it is inevitable to disperse uniformly in harmless extender. And, for example, solubility of vegetable peptide is generally much influenced by pH or concentration of salt (ion intensity), so particularly in acidic zone, solubility of it deteriorates and it deposits or coheres, ingestion by oral administration of it mixed with food is not always easy, so it is required to disperse the peptide uniformly in harmless means or dispersing means.

### BACKGROUND

1. Technical Field

The present invention relates to food ingredient comprising functional peptide extracted from coffee bean and having anti-tumor characteristic.

2. Related Art

As a technique for extracting effective ingredient contained in vegetable or the like, it is described in JP-A-H09-67259. This extracting technique is about extracting effective ingredients of small quantity from vegetable, animal, mineral, etc. The inventor established a technique for extracting effective ingredient of small quantity from coffee bean, soybean, etc.

Extract ingredient by the technique is obtainable in water solution state, however quantity of effective ingredient in water solution is very small, so it is difficult to ascertain the sort of ingredient, and inconvenient to handle, so usage of it has been restrained.

So, the inventor devised solidifying technique for extract obtained by said extracting technique and described it in JP-A-2003-117303. By this solidifying technique, ascertainment and classification of extract became easy, so the inventor found out novel functional peptide that is included in coffee-bean extract and has anti-cancer and anti-tumor characteristic, and described the novel invention in JP-A-2005-516790.

### SUMMARY

However, extract efficiency of the conventional technique is not so high. That is, so much material and long time is required for extracting. And, quantity of novel ingredient of peptide obtainable by the extracting is very small, so it is difficult to use the ingredient as it is for material of medicine or food. So, it is inevitable to disperse the ingredient uniformly in harmless extender. And, for example, solubility of vegetable peptide is generally much influenced by pH or concentration of salt (ion intensity), particularly in acidic zone, solubility of it deteriorates and it deposits or coheres, so ingestion by oral administration of it mixed with food is not always easy, so it is required to disperse the peptide uniformly in harmless means or dispersing means.

An advantage of some aspect of the invention provides A food ingredient manufactured by process comprising:
A: Step for powdering fresh milk by freeze drying;
B: Step for dissolving powered milk obtained by the step A in water solution of coffer-bean-extract peptide;
C: Step for powdering the solution obtained by the step B by freeze drying, wherein the coffee-bean-extract peptide is manufactured by process comprising:
(a) Step for generating atomized-water particles by atomizer including heater for heating reserved water to a desired temperature and atomizing means for water,
(b) Step for extracting peptide ingredient onto surface of coffee-been-crushed particle by decompressing and vibrating the atomized-water particle with material layer of the coffee-bean-crushed particles filled in extracting device,
(c) Step for capturing peptide ingredient extracted on surface of the crushed particle into the atomized-water particle by passing of the atomized-water particle with air flow through the material layer that is vibrated in decompressed state,
(d) Step for liquefying the atomized-water particle capturing the peptide ingredient by condensing device,
(e) Step for collecting by dropping liquefied peptide-ingredient-containing water into reservoir,
(f) Step for recycling atomized-water particle that is not liquefied by the condensing device to the atomizer, and
(g) Step for solidifying peptide-ingredient-containing water by contacting the peptide-ingredient-containing water with absorbent and drying the absorbent.

And, according to an aspect of the present invention, it is preferable that means for vibrating the material layer of the coffee-bean-crushed particle is magnetic vibrator or ultrasonic vibrator.

Moreover, it is preferable that material of the absorbent can include glass fiber, and the drying can be freeze drying.

Moreover, it is preferable that the coffee-bean-extract peptide can have amino acid sequence of tyrosine-glycine-serine-arginine-serine.

According to another aspect of the present invention provides a food ingredient comprising peptide having tyrosine-glycine-serine-arginine-serine and manufactured by process comprising:
(a) Step for heating water to desired temperature;
(b) Step for atomizing the heated water;
(c) Step for contacting coffee-bean-crushed particles with heated atomized-water particle with vibrating under decompressed state;
(d) Step for condensing the atomized-water particle;
(e) Step for collecting cooled condensate obtained by the step (d);
(f) Step for mixing the cooled condensate with milk; and
(g) Step for powdering mixture of the cooled condensate and the milk by drying.

And, it is preferable that the drying in step (g) can be freeze drying.

Moreover, it is preferable that the milk in step (f) can be obtained by drying fresh milk.

By uniformly dispersing efficiently-extracted-coffee-bean peptide in milk, even small quantity of peptide can be efficiently usable. And, in the milk, the peptide exists to be uniformly dispersed between caseins, colloid particles of fat globule, so it is easily digestible and absorbable, and permeability to cell is increased.

### BRIEF DESCRIPTION OF THE DRAWINGS

FIG. 1 is block diagram of manufacturing apparatus and manufacturing method.

FIG. 2 is external perspective view of manufacturing apparatus.

FIG. 3 is a perspective view showing interior of the cooling chamber comprised in the apparatus.

FIG. 4 is a external perspective view of the external cylinder included in the extracting device.

FIG. 5 is a external perspective view of the internal cylinder included in the extracting device.

FIG. 6 is a table showing the result of test 1.

FIG. 7 is a table showing the result of test 2.

FIG. 8 is a table showing the result of test 3.

### DESCRIPTION OF EXEMPLARY EMBODIMENTS

According to an aspect of the invention, the food ingredient is manufactured by mixing milk and coffee-bean-extract peptide, and the method of mixing is preferably as follows.
A: Step for powdering fresh milk by freeze drying,
B: Step for dissolving powered milk obtained by the step A in water solution of coffer-bean-extract peptide,
C: Step for powdering the solution obtained by the step B by freeze drying.

As water solution of coffee-bean-extract peptide, coffee-bean-extract peptide refined by process as follows and dissolved in water is used.
(a) Step for generating atomized-water particle by atomizer including heater for heating reserved water to a desired temperature and atomizing means for water,
(b) Step for extracting peptide ingredient onto surface of coffee-been-crushed particle by decompressing and vibrating the atomized-water particle with material layer of the coffee-bean-crushed particles filled in extracting device,
(c) Step for capturing peptide ingredient extracted on surface of the crushed particle into the atomized-water particle by passing the atomized-water particle with air flow through the material layer that is vibrated in decompressed state,
(d) Step for liquefying the atomized-water particle capturing the peptide ingredient by condensing device,
(e) Step for collecting by dropping liquefied peptide-ingredient-containing water into reservoir,
(f) Step for recycling atomized-water particle that is not liquefied by the condensing device to the atomizer, and
(g) Step for solidifying peptide-ingredient-containing water by contacting the peptide-ingredient-containing water with absorbent and drying the absorbent.

And, according to another aspect of the invention, water solution of coffee-bean-extract peptide refined by process as follows also can be used.
(a) Step for generating atomized-water particle by atomizer including heater for heating reserved water to a desired temperature and atomizing means for water,
(b) Step for extracting peptide ingredient onto surface of coffee-been-crushed particle by decompressing and vibrating the atomized-water particle with material layer of the coffee-bean-crushed particles filled in extracting device,
(c) Step for capturing peptide ingredient extracted on surface of the crushed particle into the atomized-water particle by passing the atomized-water particle with air flow through the material layer that is vibrated in decompressed state,
(d) Step for liquefying the atomized-water particle capturing the peptide ingredient by condensing device,
(e) Step for collecting by dropping liquefied peptide-ingredient-containing water into reservoir, and
(f) Step for recycling atomized-water particle that is not liquefied by the condensing device to the atomizer.

And, in the above description, temperature of water in the atomizer is approximately 80° C. and less, and it is preferable to set the temperature of material layer and atomized-water particle in extracting device to be approximately 60° C.-70° C.

Material layer of the coffee-bean-crushed particles is decompressed and vibrated. Materials are repetitively vibrated by the vibration, so the atomized-water particles uniformly contact with all surface of the material, and the surface can efficiently capture the extracted ingredient. As a means for vibrating, magnetic vibrating means, ultrasonic vibrating means, or other known vibrating means can be used.

Hereinafter, preferred embodiment of apparatus for manufacturing peptide extracted from coffee bean (containing water) will be described in detail with reference to the accompanying drawings.

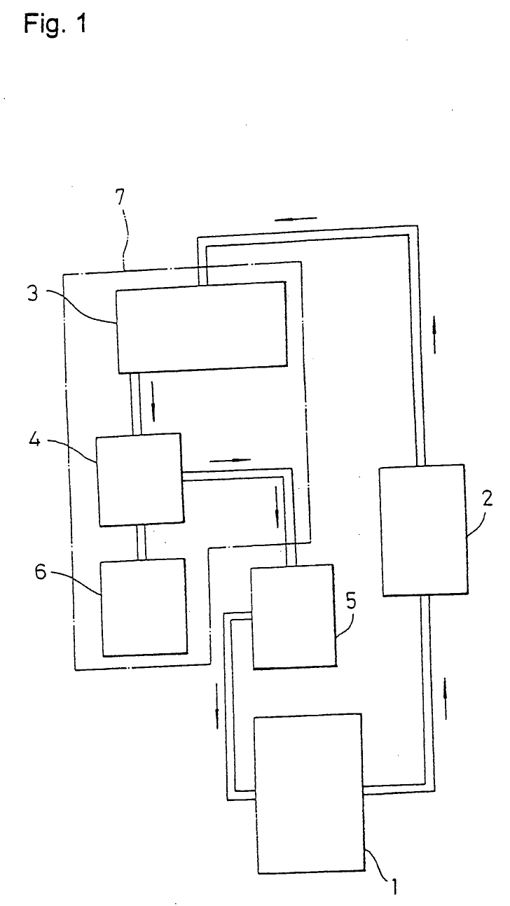

FIG. 1 is a block diagram showing constitutions of a first embodiment of the manufacturing device, and in the FIG. 1, reference numeral symbol 1 is an atomizer, reference numeral symbol 2 is an extracting device for extracting effective ingredient from raw material of crushed coffee bean with vibration by atomized-water particle sent from the atomizer 1, reference numeral symbol 3 is a condensing device for liquefying the atomized-water particle holding the effective ingredient of the raw material transferred from the extracting device 2, reference numeral symbol 4 is a reservoir tank for receiving water liquefied at the condensing device 3 and containing the effective ingredient of the raw material from the condensing device 3, reference numeral symbol 5 is a blower provided between the reservoir tank 4 and the atomizer 1. And, reference numeral symbol 6 is a second reservoir tank connected to the reservoir tank 4, and reference numeral symbol 7 is a cooling means for cooling the condensing device, reservoir tank 4, and the second reservoir tank 6. As shown in the figure, each device such as the atomizer 1, the extracting device 2 is respectively connected by connecting pipe, so that a circulating path is formed around the atomizer 1, and it is made such that the atomized-water particle circulates together with air flow by operation of the blower in this circulation path.

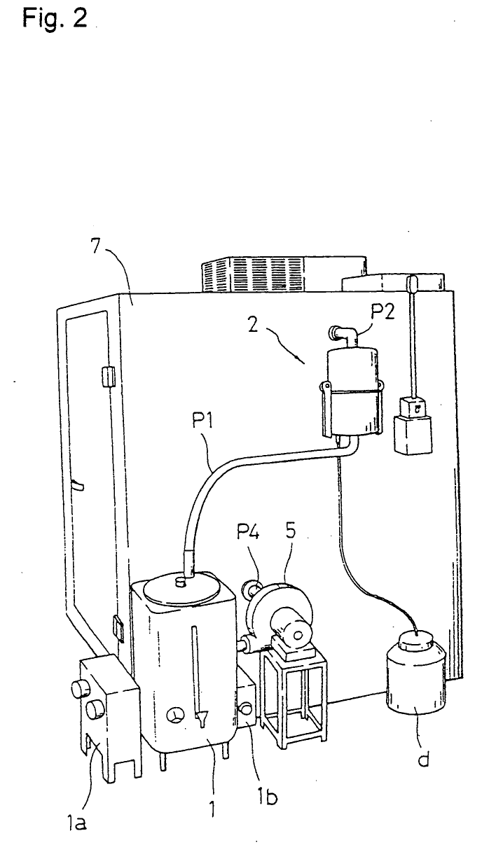

FIG. 2 is a external perspective view of the manufacturing apparatus comprising above described constitutions. In the FIG. 2, reference numeral symbol 1 is an atomizer structured with a water tank that is made of stainless steel and has width and length of 35 cm and depth of 60 cm, and the water tank is made such that approximately 30-40 liters of water is always reserved in this water tank during operation. Reference numeral symbol 1a is a ultrasonic-wave-generating device, and eight vibrators included in the device la are provided at bottom portion of the water tank 1, and each vibrator has an ability to atomize water of about 0.5 liter of per one hour. Reference numeral symbol 1b is a heater for maintaining the water temperature to a desired temperature.

And, reference numeral symbol 2 is hereinafter described extracting device, and it is provided at a side wall of the cooling chamber 7 of cooling means, and connected with the atomizer 1 through a flexible plastic pipe P of 28 mm in diameter and about 1.3 m in length. Further, reference symbol d is a discharged-water tank receiving water discharged from the extracting device 1. And, reference symbol P2 is a metal pipe of 40 mm in diameter for connecting the extracting device 2 and a hereinafter described condensing device 3.

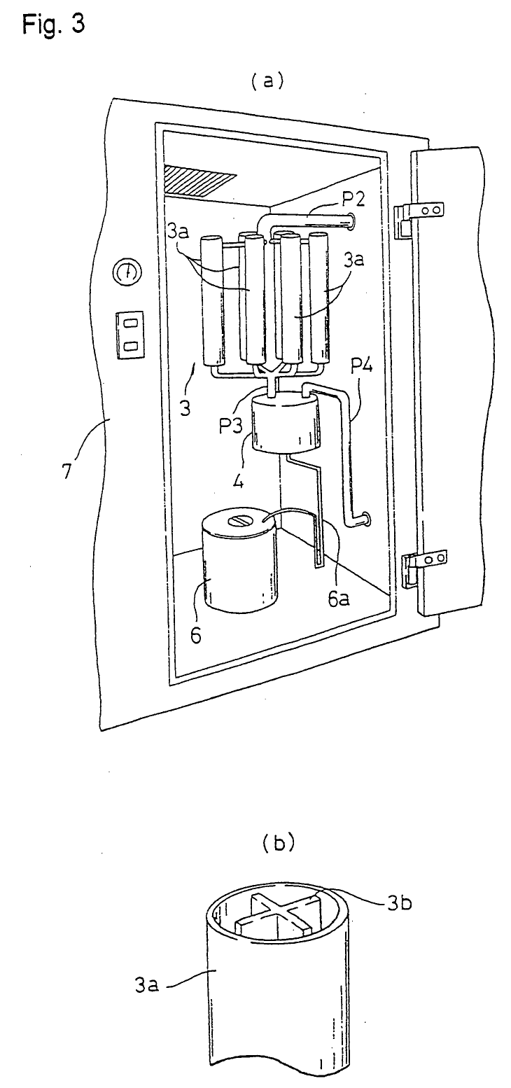

FIG. 3(a) is a fragmentary perspective view showing an interior of the cooling chamber 7, and in the FIG. 3(a), reference numeral symbol 3 is a condensing device made of a plurality (six in this embodiment) of condensing cylinders 3a, and connected with the extracting device 2 provided at outside of the cooling chamber 7 through the pipe P2 as described above. In this embodiment, each condensing cylinder 3a is constituted by metal pipe of 85 mm in diameter and about 550 mm in length, and a cooling plate 3b is provided at interior of each condensing cylinder 3a as shown in FIG. 3(b). Top end of each condensing cylinder 3a is connected to the connecting pipe P2 through a branch pipe, and bottom end them are connected to a connecting pipe P3 through a branch pipe.

Reference numeral symbol 4 is a reservoir tank connected with the condensing device 3 through a connecting pipe P3 of 40 mm in diameter, and the reservoir tank is provided for receiving water liquefied from the atomized-water particle by the condensing device 3. Upper portion of this reservoir tank 4 and the blower 5 provided at outside of the cooling chamber are connected through a connecting pipe P4 of 40 mm in diameter. Further, reference numeral symbol 6 is a second reservoir tank, and it is connected with the reservoir tank 4 through a drain pipe 6a. The cooling device of the cooling chamber 7 is provided at top ceiling portion, however a window type air conditioner may be fixed at side wall portion for increasing cooling capacity.

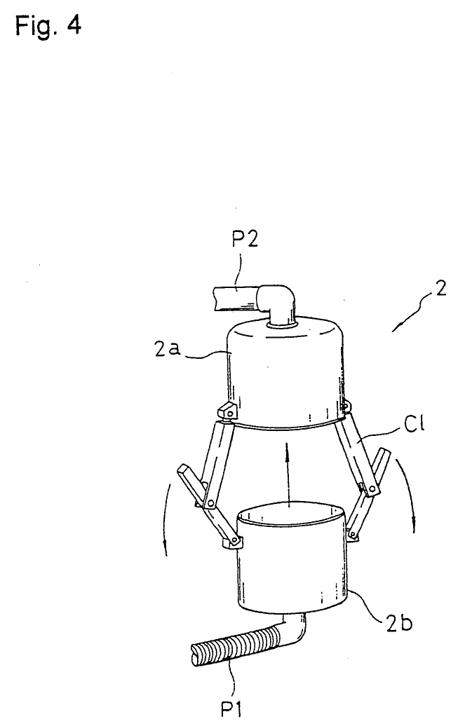

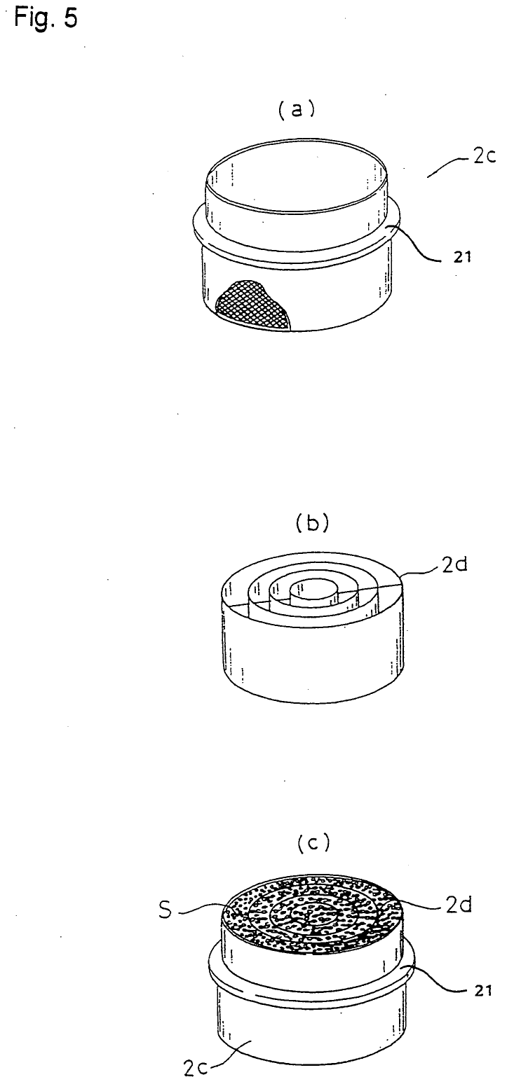

FIG. 4 is a external perspective view of external cylinder that is a constituting element of the extracting device 2, and the external cylinder includes a first external cylinder 2a and a second external cylinder 2b, and both external cylinders are made to be supported by clamping device C1 such that they freely joint, release, and open. And, they are formed to be cylindrical shape of 200 mm in diameter and about 150 mm in depth, and they are made of stainless steel. Further, a temperature sensor for detecting temperature during extracting operation is installed at the second external cylinder 2b provided at lower side. FIG. 5 is a explanatory view of internal cylinder that is constituting element of the extracting device 2, and FIG. 5(A) is a perspective view of the internal cylinder 2c. The internal cylinder 2c has shape and size capable of fitting to the external cylinder, and a net portion for holding the raw material crushed to small pieces is provided at bottom portion, and vibrating base as a vibrating means is provided at side portion. FIG. 5(b) shows a guide plate for being inserted into the internal cylinder 2c, and as shown in FIG. 5(c), it is made to partition the crushed pieces S of coffer bean in an interior of the internal cylinder 2c. Existence of this guide plate 2d bring an effect to make passage of the pulverized minute particles to be easy and smooth as will be described hereinafter. And, this guide plate 2d may be formed in spiral shape. As described above, the extracting device 2 is constituted with a pair of external cylinder and the internal cylinder for being inserted to that.

Operation of manufacturing apparatus and manufacturing process for coffee-bean-extract peptide (containing-peptide water) will be described based on the above described constitution. In the embodiment, raw coffee bean is used as raw material. First, coffee beans crushed to a magnitude of particulate are filled in the internal cylinder 2c shown in FIG. 5(a). The weight of coffee beans filled in the internal cylinder is about 1800 g. With the coffee beans filling, guide plate 2d shown in FIG. 5(b) is installed in the internal cylinder 2c. Further, after filling, if a net is provided on the coffee beans, the coffee beans may be stably maintained in the internal cylinder.

Next, the internal cylinder 2c is inserted into the external cylinder 2 shown in FIG. 4. On the other hand, water of about 30-50 liters is reserved in the atomizer 1 shown in FIG. 2. And, it is constituted such that aforementioned amount of water is always automatically maintained in the atomizer 1. When preparation of water into the atomizer 1 and raw material of coffee beans into the extracting device 2 is finished, temperature of water in the water tank 1 is set by the heater 1b of the atomizer 1. When coffee beans are used as raw material, the set temperature of 85° C. seems to be most preferable by experience. The temperature of 85° C. is most suitable for maintaining temperature in the extracting device 2 to be 60-70° C., as will be described hereinafter.

When the temperature of water in the water tank 1 reaches the set temperature of 85° C., switches of the ultrasonic wave generating device la and the blower are turned on. By operation of the blower 5, air flow circulates the circulation path formed by the atomizer 1, the extracting device 2, the condensing device 3, the reservoir tank 4, the blower 5, and the connecting pipes connecting these respective devices. By this configuration, the atomized-water particle generated by the atomizer 1 passes through the aforementioned plastic pipe P1 together with the air flow and reaches the extracting device 2. Further, the temperature of the atomized-water particle in the extracting device 2 is preferable to be in the range of 60-70° C., as described above. For this reason, the temperature in the extracting device is always detected by the temperature sensor provided to the extracting device 2, and the water temperature in the atomizer 1 is adjusted in response to the detected result by the sensor in order to obtain preferable temperature.

As described above, the air flow circulates through each device by operation of the blower 5, however crushed coffee beans as raw material are filled in the extracting device, so air flow passing through the pipe P1 suffers resistance, and the flow weakens. On the other hand, at downstream side of the connecting pipe P2, nothing disturbs the air flow. So, space in the extracting device 2 becomes decompressed state.

When the space in the extracting device 2 become decompressed state, known ingredient and unknown ingredient contained within the coffee beans are extracted out to the surface of crushed pieces of coffee beans that are raw material. The above described various ingredients extracted to surface of the crushed coffee beans are captured by the atomized-water particles that pass through. And, the crushed coffee beans are vibrated by the vibrating means 21, atomized-water particles uniformly contact with all surfaces of the crushed coffee beans and capture ingredients. And, as described above, since the temperature in the extracting device, more particularly in the internal cylinder 2c is maintained to be approximately 65° C., ingredient contained in the coffee beans are extracted into the atomized-water particle without being destroyed by heat.

The atomized-water particles containing the effective ingredient of coffee beans reach the respective condensing cylinder 3a of the condensing device 3 through the connecting pipe P2 together with the air flow. Since the condensing cylinder 3a and the cooling plate 3b therein are present in the cooling chamber 7 for cooling, the atomized-water particles passing through therein are liquefied and changed to water containing effective ingredient of coffee bean. This water containing ingredient of coffee bean is dropped into the reservoir tank 4, and finally collected to the second reservoir tank 6 through the drain pipe 6a. The coffee-bean-ingredient-containing water collected to the second reservoir tank 6 is filtered by a filter for eliminating miscellaneous impurities, and then extract-ingredient-containing water as final product in which effective ingredients of coffee bean are contained as major ingredient is made.

In the meanwhile, the atomized-water particles which were not liquefied at the condensing device 3 are sucked to the blower 5 through the connecting pipe 4 together with the air flow and return to the atomizer 1, and thereafter are sent to the extracting device 2 again through the plastic pipe P1.

As described above, the atomized-water particles circulate the circulating path so that the effective ingredient of the coffee bean as a raw material is captured by the atomized-water particles, and thereby the extract-ingredient-containing water containing effective ingredient of the coffee bean is obtained by liquefying the atomized-water particles, here the single operation period of the manufacturing apparatus is one hour. That is, in the above described embodiment, extract-ingredient-containing water of about 3˜4 liters can be finally manufactured by executing one hour extraction with about 180 g of crushed coffee bean pieces.

However, although very small-sized particulates of coffee bean are used in the above described embodiment, concentration of the effective ingredient contained in the final product can be adjusted by changing the size of the crushed pieces of the coffee bean. That is, as the crushed piece of the coffee bean is made to be finer, product of higher concentration can be obtained. However, in that case, the manufacturing quantity per hour is decreased. On the contrary, when the crushed piece is made to be bigger, the manufacturing quantity per hour is increased, and the concentration of ingredient becomes lower.

In the above described embodiment, although the guide plate 2d is used in the internal cylinder 2c of the extracting device 2, when this guide plate is used, yield quantity of extract-ingredient-containing water per hour increases about 20% compared to a case of no use, but concentration is decreased. And, by vibrating raw material of coffee bean, efficient extract is possible, so same quantity of extract-ingredient-containing water can be obtained from a quarter quantity of raw material.

However, in the embodiment related to the above described manufacturing apparatus, as shown in FIGS. 1, 2, and 3, although non-liquefied atomized-water particles are returned to the atomizer 1 through the connecting pipe P4 and the blower 5, since this atomized-water particles are cooled at the cooling chamber 7, so temperature of the atomized-water particles goes down to about 15° C. And, when this cooled atomized-water particles are mixed with newly atomized-water particles generated at the water tank 1 in this state and sent to the plastic pipe P1, temperature of the newly atomized-water particle goes down and condenses to water droplet, so transfer of the atomized-water particle in the pipe P1 may be disturbed. To solve this problem, it is preferable to heat some part of the connecting pipe P4 provided at outside of the cooling chamber, or to rotate the atomized-water particle sent from the blower 5 to the atomizer 1 with rectifying plate of spiral shape, and then temperature of the atomized-water particle is raised during the rotation, and after that, the atomized-water particle is preferably sent to the plastic pipe P1.

Meanwhile, water comprising peptide extracted from coffee bean by above described manufacturing apparatus is mixed with fresh milk to obtain novel food ingredient. That is, coffee-bean-extract peptide uniformly spread and stably exists in milk ingredient, so the coffee-bean-extract peptide exists even in very small quantity of milk, moreover the peptide is mixed with milk, so the food ingredient has high compatibility with peptide transporter in viscera and arrives at desired viscera in state of easily absorbable and easily permeates into cells.

However, this liquefied food ingredient is inconvenient to preserve and handle, so powder or solid type is preferable as a final product.

In the embodiment, fresh milk is powdered by known freeze drying, and the powdered milk is dissolved in water solution of coffee-bean-extract peptide obtainable by above described embodiment, moreover the mixture of powdered milk and the water solution of peptide is powdered by freeze drying, and food ingredient of powder type is made. And, known nonfat dry milk can be used instead of the powdered milk.

In the above described embodiment, although water solution of coffee-bean-extract peptide obtained by extracting of coffee bean is used, mixture of solidified coffee-bean-extract peptide and water also can be used.

That is, by contacting coffee-bean-extract-peptide-containing water obtained by above described manufacturing apparatus with absorbent, thereafter by drying the absorbent, the coffee-bean-extract peptide is solidified.

First embodiment of procedure for solidification is as follows. First of all, as for the absorbent, non-nutritional material is used. Suitable materials for the absorbent is hydrophilic membrane filters such as polyvinylidene fluoride membrane of the Durapore filter commercially available from Milipore Corporation. And, membrane made of glass fiber, cotton, nylon, cellulose, or paper material that is used for tea bag is also desirable. The shape of the membrane is not particularly limited, and can be sheet shape, disc shape, etc.

The absorbent contacts with the extract (coffee-bean-extract peptide) obtained by above manufacturing apparatus. It is preferable that the entire surface of the absorbent is wetted by the liquid final product containing the extract. When membrane is used as absorbent, to completely wet the membrane with the final product (extract), for example, driving force of vacuum pump or the like can be used to push or pull the extract through the filter. Optionally, the absorbent can be heated before or during wetting to expand pores and enhance the wetting. Alternatively or in addition to the heating, the final product (extract) may be heated. If the absorbent is sufficiently wetted by the final product (extract), the extract more preferably adheres to the absorbent than in dried condition. Drying can be done by freeze drying, heat drying, air flow drying, however freeze drying is preferable. Dried extract can be preserved for a long time without deterioration. And, the dried extract can be dissolved in water or other solvent to obtain liquid containing effective ingredient. Pressure can be applied to facilitate the dissolution, if desired.

The inventor find out that coffee-bean-extract peptide obtained by above described embodiment is pentapeptide that has amino acid sequence of: tyrosine-glycine-serine-arginine-serine, and the coffee-bean-extract peptide has anti-cancer, anti-inflammation, anti-tumor characteristic.

Composition containing effective amount of the coffee-been-extracted peptide are useful as drug or food ingredient for human or animal, for example, useful in treatment and/or prevention of various diseases and conditions including anticancer and anti-inflammatory condition. Various diseases against which the peptide is effective include cancers of various types. Various types of inflammation against which the peptide is effective include encephalitis, cerebral meningitis, marginal blepharitis, conjunctivitis, keratitis, iritis, retinitis, stomatitis, cheilitis, glossitis, tonsillitis, internal otitis, external otitis, glossitis, tonsillitis, internal otitis, external otitis, otitis media, gastritis, duodenitis, pneumonia, pleurisy, bronchitis, rhinitis, colitis, inflammation of the small intestine, nephritis, pyelitis, pancreatitis, cholecystitis, hepatitis, thyroiditis, prostatitis, cystitis, myotis, periostitis, osteomyelitis, orchitis, endometritis, vaginitis, ovaritis, dermatitis, arthritis, periproctitis, lymphadenitis, diabetes (inflammation of the pancreatic islets), common cold (tonsillitis, bronchitis, rhinitis, mucositis), urticaria, various kinds of eczema (dermatitis), nephrosis (nephritis), alveolar pyorrhea (parodontitis, aplicalis, endodontitis), asthma (bronchitis), neuralgia (neuritis), infectious diseases (inflammation induced by bacteria and virus), allergy (inflammation induced by antigen-antibody reaction), leprosy (viral dermatitis, and myotis), cancer (inflammation and fibroid induration are also causes), ulcer (progression of inflammation), fibroid induration (progression of inflammation and ulcer), reduced energy (adenitis), keratosis, collagen diseases, hysteria, neurosis, liver cirrhosis, hypertension, thrombosis, angina, rheumatism, gout, stiffness, Alzheimer, Lyme disease, mad cow disease, and inflammation due to parasites.

The coffee-bean-extract peptide having above described amino acid sequence is isolated and classified as follows.

Coffee bean as a raw material is used in above described extraction method. With the obtained extract, glass fiber membrane of 96.4 g is wetted. The membrane is extracted three times with ethyl acetate of 300 ml. The ethyl acetate is almost dried under vacuum by rotary evaporator. Temperature of solvent layer does not exceed 40° C. Residue is liquid (20.6 ml) with light brown color.

Then, the extract is extracted with ethyl ether of 150 ml. Layer of the ethyl ether is dried with anhydrous sodium sulfate. The sodium sulfate is removed and the ethyl ether layer is dried in rotary evaporator under vacuum. White compound of short-needle shape is obtained. This compound is dissolved in ethanol and re-crystallized by evaporation of the ethanol. The compound is chemically analyzed, and we obtain following parameters:

| | |
|---|---|
| Absorption Spectrum: | 348 mu |
| Thin layer chromatography | 3.8 |
| Silica gel by Merk Corp.: | |
| Micro Kjeldahl: | 12.4% |
| Melting Point: | 172° C. |
| Amino acid sequence: | Tyrosine-Glycine-Serine- |
| | Arginine-Serine |

### Test 1

### Protocol Design:

Adjuvant-induced arthritis model has been developed by using rats to allow screening of compound that may be useful in treatment of rheumatoid arthritis of human. Adjuvant-induced arthritis responds to both of steroids and non-steroid. Degree of inflammation is estimated by measuring differences in weight and/or volume of foot.

### Test Organism:

Rats with weight of 150-200 g have been purchased from Animal Technologies Ltd., Kent, Wash. They are male Sprague-Dawley rats. The rats have been kept in stainless steel cages individually with free water and food (Harlan Teklan Rodent Diet). Light and darkness cycle has been maintained to be 12 hours of light and 12 hours of darkness. Temperature has been maintained to be 22° C.±3° C. with relative humidity of from 40% to 70%.

### Dose Administration:

Test material is dissolved or suspended in deionized water at doses of 10 mcg/kg or 1 mcg/kg to body weight. The test compound and hydrocortisone are administered by gavage.

### Experimental Design:

Male Sprague-Dawley rats (150-200 g) are sensitized by injecting Fruend complete adjuvant (0.5% suspension of killed mycobacterium tuberculosis (H37RA, Difco in mineral oil)). Aliquot of 0.1 ml is intradermally administered at plantar portion of right rear leg of each rat.

The test materials are orally administered (by gavage) to 5 rats in each treatment group once per single day during 10 days. Administration of the test materials initiates from sensitization day.

The left rear paw was investigated just before sensitization and again on tenth day. Plantar-edema-inhibitory rate and body-weight-increase rate are determined by comparing with non-sensitized rat groups.

Weights of the paws are averaged. Anti-inflammatory activity is determined by comparing weights of paws and calculated as follows:

% Anti-inflammatory response=[(Mean paw weight of controll group)−(Mean paw weight of test group)]/(Mean paw weight of test group)

Hydrocortisone is used as a positive control. The hydrocortisone is a general anti-inflammatory administered to rheumatoid arthritis patients at a dose of 10 mg/kg to body weight. Peptide is administered by 10 mcg/kg or 1 mcg/kg to body weight.

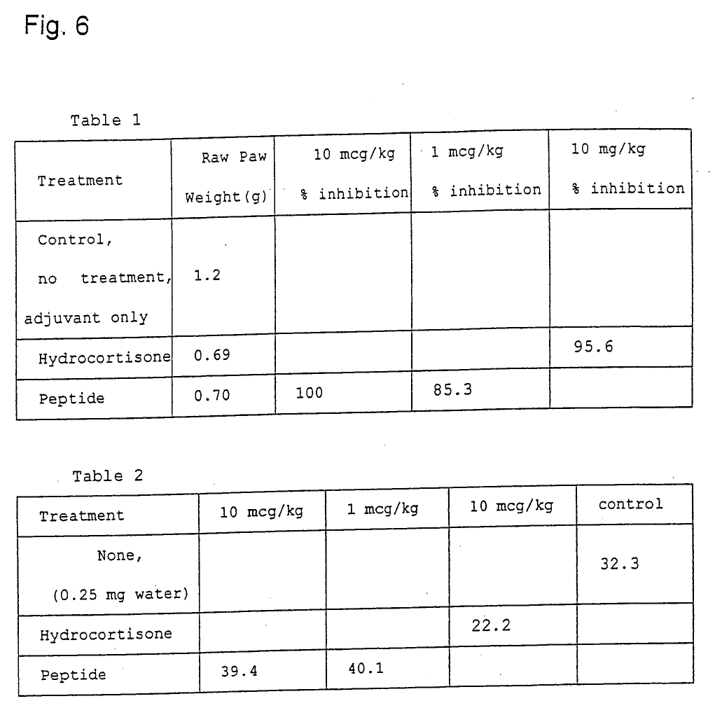

As shown in table 1 and 2 of FIG. 6, the result indicates that the peptide of 10 mcg/kg to body weight inhibits the induced inflammation 100%. At level of 1 mcg/kg to body weight, the peptide inhibits 85.3%.

Accordingly, the peptide is powerful inflammation inhibitor without inducing weight loss.

Test 2
(a) Test Organism

| Species: | Mouse |
|---|---|
| Strain: | Swiss-Webster |
| Supplier: | Animal Technologies Ltd., Kent, WA |
| Sex: | Female |
| Weight: | 26~30 |
| Number Used: | 60 |

### (b) Husbandry

Research Facility: USDA Registration No. 91-R-043. NIH Public Health Assurance No.A3932-01

Animal Rooms: Light cycle—12 hours light, 12 hours darkness. Temperature/Relative humidity: every attempt is made to maintain temperature of 22° C.±3° C. and relative humidity of from 40% to 70%.

Housing: Five mice per single group are kept in standard cage, according to the “Guide for the Care and Use of Laboratory Animals” of the Institute of Laboratory Resources, National Research Council.

Sanitation: Waste materials are removed twice a week. Cages and feeders are sanitized by every two weeks.

Food: Marlan Teklad Rodent Diet #8604 properly.

Food Analysis: There are no contaminants that are reasonably expected to be present in the diet material and known to be capable of interfering with the purpose or conduct of the investigation.

Water: Deionized water available for living thing and not containing pyrogen.

Water analysis: The system is periodically maintained by Continental Water System Company by every six months (change carbon tanks, D.I. beds and in-line filters). Replacement of the UF membranes is by every two years, the UV lamp by every year.

Test Article: Isolates form coffee bean.

Dose Administration: Water solution of the isolate is administered by gavage for continuous eleven days.

Administration Volume of Test Article: 0.2 ml by gavage.

Treatment Time: For eleven days. Sacrifice on 12^{th} day.

### (c) Assay Method

The tumor stock is Sarcoma 180 that originates in the laboratory of American Type Culture Collection. This stock culture has been passed at weekly intervals as ascites in non-treated Swiss-Webster mice.

The studies are all conducted with Swiss-Webster mice obtained from Animal Technologics Ltd., Kent, Wash. To prepare the inoculum, ascites fluid of 7^{th}˜12^{th} day mouse is aspirated by sterile technique. Viability of tumor cells is checked by trypan-blue staining technique. After number of cells is confirmed, the tumor cells are diluted with normal saline or phosphate buffered saline to obtain final concentration of from 1×10⁶ to 2×10⁶ cells per mm³. Then the tumor suspension is injected into mice. The final dilution is plated on trypticase-soy agar to find out whether there is contamination or not.

One tenth (0.1 ml) of the suspension is inoculated into left rear leg muscle (hamstring muscle mass) of each mouse. The inoculated mice are placed into one large cage and then are randomly segregated into groups of five mice. The mice are housed in shoebox cage with wood shavings therein in condition of free access to water and laboratory chow. The mice are weighed on inoculation day, on 7^{th} day, on 12^{th} day, and at the time of sacrifice. Treatment for the mouse begins the day of transplantation. At the end of observation, the mice are sacrificed with ether anesthesia. Skin on the left rear leg is removed to expose the tumor, and then the leg and tumor are picked out to the hip joint. Remnant skin is removed and the legs with tumors are weighed individually. Ten normal legs (right legs) are prepared in a similar manner and weighed. Mean value of the normal legs is subtracted from the weight of the leg with tumor to obtain estimated value of actual tumor weight.

% Inhibition=[(Mean Tumor Weight (Test Group))/(Mean Tumor Weight (Control Group))]×100

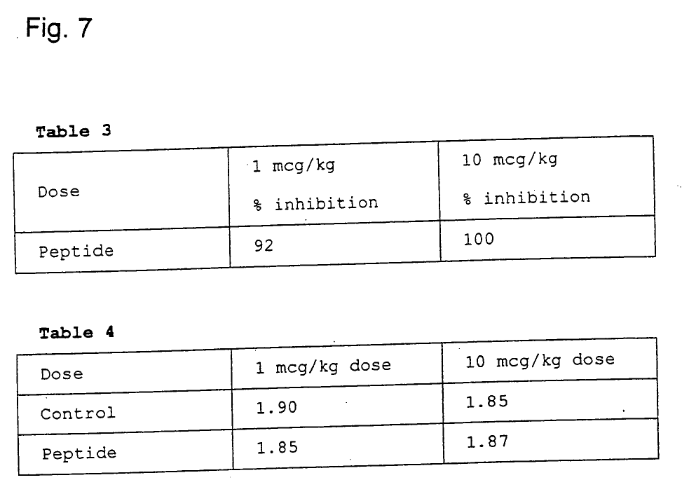

Result: Shown in Table 3, 4 of FIG. 7.

As shown in table 3 and 4, the result indicates that the peptide of 10 mcg/kg to body weight proves 100% inhibits. Increase of weight by administration of peptide proves nontoxic like the control.

Test 3
(a) Test Organism

| Species: | Mice |
|---|---|
| Strain: | Swiss-Webster |
| Supplier: | Harlan Laboratories, Inc., Gilroy, CA. |
| Sex: | male |
| Weight: | 17~20 |
| Number Used: | 10 |

### (b) Husbandry

Research Facility: USDA Registration No. 91-R-043. NIH Public Health Assurance No.A3932-01

Animal Rooms: Light cycle—12 hours light, 12 hours darkness. Temperature/Relative humidity: every attempt is made to maintain temperature of 22° C.±3° C. and relative humidity of from 40% to 70%.

Housing: Five mice per single group are kept in standard cage, according to the “Guide for the Care and Use of Laboratory Animals” of the Institute of Laboratory Resources, National Research Council.

Sanitation: Waste materials are removed twice a week. Cages and feeders are sanitized by every two weeks.

Food: Harlan Teklad Rodent Diet #8604 properly and other acceptable Lab chow.

Food Analysis: There are no contaminants that are reasonably expected to be present in the diet material and known to be capable of interfering with the purpose or conduct of the investigation.

Water: Deionized water available for living thing and not containing pyrogen

Water analysis: The system is periodically maintained by Continental Water System Company by every six months (change carbon tanks, D.I. beds and in-line filters). Replacement of the UF membranes is by every two years, the UV lamp by every year.

Test Article: Mixture of coffee-bean-isolated peptide (YGSRS, amino acid sequence: tyrosine-glycine-serine-arginine-serine) and nonfat dry milk (the peptide is diluted in solution including nonfat dry milk of 1 g and distilled water of 10 ml).

Dose Administration: 1 ml/day/mouse (two times with 0.5 ml in a day), for continuous fourteen days right after transplantation of tumor by gavage.

### (c) Experimental Design

Goroup 1. five mice are treated with tumor in distilled water two times in a day.

Goroup 2. five mice are treated with tumor and mixture of coffee-bean-isolated peptide (YGSRS, amino acid sequence: tyrosine-glycine-serine-arginine-serine) and nonfat dry milk two times in a day.

The test article of 1 mm is treated per a day. Dose of 0.5 ml is administered in the morning and 0.5 ml in the afternoon at approximately same time of each day. Treatment continues for fourteen days starting from right after transplantation of tumor.

At the end of 14^{th} day of gavage, the mice are sacrificed and the tumors are weighed.

### (d) Assay Method

Sarcoma 180 tumor is used to furnish tumor cells with livability of 2×10²/0.1 ml that is made up from transplant-source mouse, and the tumor cells are injected into left rear leg muscle (hamstring muscle mass) of mouse.

At the termination of the experiment, the mouse is weighed and the left rear leg is amputated at thigh. And, skin is removed to expose the tumor portion. Net tumor weight is determined by subtracting the mean value obtained from ten normal legs.

### (e) Result

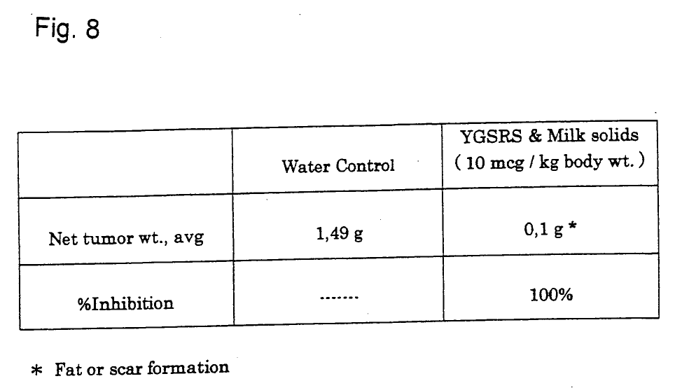

The result is as shown in table 5 of FIG. 8. As for table 5, tumor inhibition percent is calculated compared to the water control values. All administration is oral by gavage.

### (f) Conclusion

The mixture of coffee-bean-isolated peptide and nonfat dry milk actively functions to inhibit the sarcoma 180 tumor in condition of every single day administration with 10 mcg/kilo to body weight for fourteen days.

What is claimed is:

1. A food ingredient manufactured by process comprising:

A: Step for powdering fresh milk by freeze drying;

B: Step for dissolving powered milk obtained by the step A in water solution of coffer-bean-extract peptide;

C: Step for powdering the solution obtained by the step B by freeze drying, wherein the coffee-bean-extract peptide is manufactured by process comprising:

(a) Step for generating atomized-water particles by

What is claimed is:

1. A food ingredient manufactured by a process comprising:
powdering fresh milk by freeze drying;
dissolving the powdered milk in water solution of coffee-bean-extract peptide;
powdering the solution by freeze drying, wherein the coffee-bean-extract peptide is manufactured by a process comprising:
generating atomized-water particles by atomizer including heater for heating reserved water to a desired temperature and atomizing means for water,
extracting peptide ingredient onto surface of coffee-been-crushed particle by decompressing and vibrating the atomized-water particle with material layer of the coffee-bean-crushed particles filled in extracting device,
capturing peptide ingredient extracted on surface of the crushed particle into the atomized-water particle by passing of the atomized-water particle with air flow through the material layer that is vibrated in decompressed state,
liquefying the atomized-water particle capturing the peptide ingredient by condensing device,
collecting by dropping liquefied peptide-ingredient-containing water into reservoir,
recycling atomized-water particle that is not liquefied by the condensing device to the atomizer, and
solidifying peptide-ingredient-containing water by contacting the peptide-ingredient-containing water with absorbent and drying the absorbent.

2. A food ingredient according to claim 1, wherein means for vibrating the material layer of the coffee-bean-crushed particle is a magnetic vibrator or an ultrasonic vibrator.

3. A food ingredient according to claim 1, wherein material of the absorbent includes glass fiber, and the drying is freeze drying.

4. A food ingredient according to claim 1, wherein the coffee-bean-extract peptide has an amino acid sequence of tyrbsine-glycine-serine-arginine-serine.

5. A food ingredient comprising peptide having tyrosine-glycine-serine-arginine-serine and manufactured by a process comprising:
heating water to a desired temperature;
atomizing the heated water;
contacting coffee-bean-crushed particles with heated atomized-water particles with vibrating under decompressed state;
condensing the atomized-water particles;
collecting cooled condensate;
mixing the cooled condensate with milk; and
powdering mixture of the cooled condensate and the milk by drying.

6. A food ingredient according to claim 5, wherein the drying is freeze drying.

7. A food ingredient according to claim 5, wherein the milk is obtained by drying fresh milk.
